(19) United States
(12) Patent Application Publication
(10) Pub. No.: US 2011/0224261 A1
(43) Pub. Date: Sep. 15, 2011

(54) CRYSTALLINE FORM OF 4-(5--4-METHYL-4H-1,2,4-TRIAZOL-3-YL)PYRIDINE

(75) Inventors: Hans Åström, Sodertalje (SE); Alexander Minidis, Sodertalje (SE); Veronica Profir, Sodertalje (SE)

(73) Assignee: ASTRAZENECA AB, Sodertalje (SE)

(21) Appl. No.: 13/058,436

(22) PCT Filed: Aug. 11, 2009
(86) PCT No.: PCT/SE2009/050927

§ 371 Date: Apr. 29, 2011

Related U.S. Application Data
(60) Provisional application No. 61/088,049, filed on Aug. 12, 2008.

(51) Int. Cl. C07D 413/14; A61K 31/4439; A61P 1/00; A61P 1/04; A61P 23/02; A61P 25/08; A61P 25/22; A61P 25/28; A61P 25/06; A61P 9/00; A61P 25/18; A61P 25/16; A61P 19/02

(52) U.S. Cl. 514/340; 546/272.4

(57) ABSTRACT

The present invention relates to a novel crystalline form of 4-(5-{(IR)-1-[5-(3-chlorophenyl)isoxazol-3-yl]ethoxy}-4-methyl-4H-1,2,4-triazol-3-yl)pyridine. Further, the present invention also relates to the use of the novel crystalline form for the treatment of gastrointestinal disorders, pharmaceutical compositions containing it.

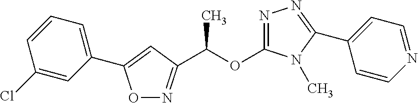

### FIELD OF THE INVENTION

The present invention relates to a novel crystalline form of 4-(5-{(1R)-1-[5-(3-s chlorophenyl)isoxazol-3-yl]ethoxy}-4-methyl-4H-1,2,4-triazol-3-yl)pyridine possessing unexpectedly favourable characteristics. Further, the present invention also relates to the use of the novel crystalline form for prevention or treatment of a mGluR5 receptor-mediated disorder, such as a neurological, psychiatric or a gastrointestinal disorder. The invention also provides pharmaceutical compositions containing it as well as processes for the preparation of the novel crystalline form.

### BACKGROUND OF THE INVENTION

The compound 4-(5-{(1R)-1-[5-(3-chlorophenyl)isoxazol-3-yl]ethoxy}-4-methyl-4H-1,2,4-triazol-3-yl)pyridine is described in WO2007/040982.

### BRIEF DESCRIPTION OF THE DRAWINGS

FIG. 1 is an X-ray powder diffractogram of 4-(5-{(1R)-1-[5-(3-chlorophenyl)isoxazol-3-yl]ethoxy}-4-methyl-4H-1,2,4-triazol-3-yl)pyridine, modification A.

### DESCRIPTION OF THE INVENTION

It has surprisingly been found that 4-(5-{(1R)-1-[5-(3-chlorophenyl)isoxazol-3-yl]ethoxy}-4-methyl-4H-1,2,4-triazol-3-yl)pyridine can exist in a novel crystalline form possessing unexpectedly favourable characteristics. The novel crystal form for the first time disclosed is hereinafter referred to as 4-(5-{(1R)-1-[5-(3-chlorophenyl)isoxazol-3-yl]ethoxy}-4-methyl-4H-1,2,4-triazol-3-yl)pyridine, modification A. The novel crystalline form can be characterized by its X-ray powder diffraction pattern, and in particular its d-spacing value of 4.0 Å.

It is thus an object of the present invention to provide a crystalline form of the neutral form of 4-(5-{(1R)-1-[5-(3-chlorophenyl)isoxazol-3-yl]ethoxy}-4-methyl-4H-1,2,4-triazol-3-yl)pyridine with advantageous properties.

It is an aspect of the present invention to provide 4-(5-{(1R)-1-[5-(3-chlorophenyl)isoxazol-3-yl]ethoxy}-4-methyl-4H-1,2,4-triazol-3-yl)pyridine modification A.

4-(5-{(1R)-1-[5-(3-chlorophenyl)isoxazol-3-yl]ethoxy}-4-methyl-4H-1,2,4-triazol-3-yl)pyridine modification A is characterized in providing an X-ray powder diffraction pattern, exhibiting substantially the following main peaks with d-values (d-value: the spacing between successive parallel hkl planes in a crystal lattice):

| d-spacing value (Å) | Relative intensity |
|---|---|
| 12.63 | Weak |
| 11.41 | Weak |
| 6.35 | Strong |
| 6.21 | Medium |
| 5.72 | Strong |
| 5.08 | Strong |
| 4.0 | Very strong |

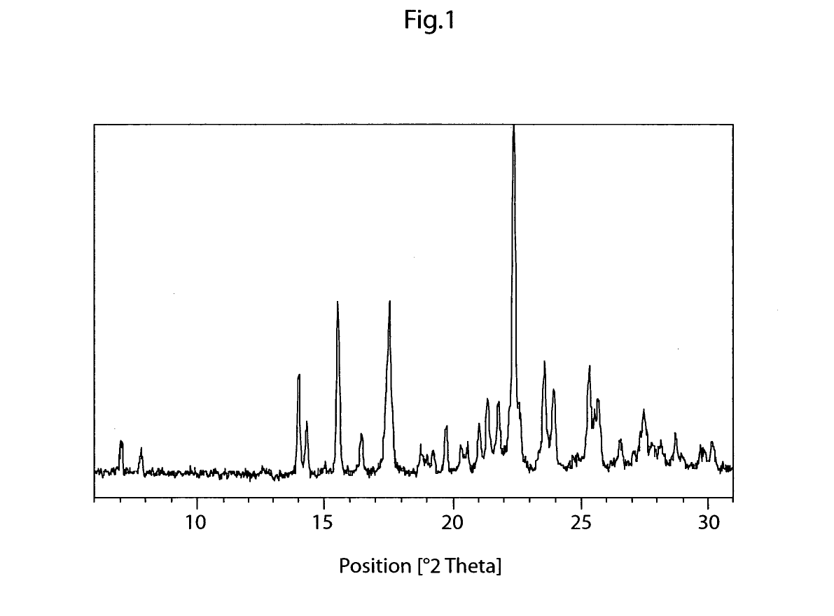

The peaks, identified with d-values calculated from the Bragg formula and intensities, have been extracted from the diffractogram of 4-(5-{(1R)-1-[5-(3-chlorophenyl)isoxazol-3-yl]ethoxy}-4-methyl-4H-1,2,4-triazol-3-yl)pyridine modification A. Only the main peaks, that are the most characteristic, significant, distinct and/or reproducible, have been tabulated (a number of weak peaks have been omitted. Peaks are only listed up to 35 degrees 2θ), but additional peaks can be extracted, using conventional methods, from the diffractogram. The presence of these main peaks, reproducible and within the error limit, is for most circumstances sufficient to establish the presence of said crystal modification. 4-(5-{(1R)-1-[5-(3-chlorophenyl)isoxazol-3-yl]ethoxy}-4-methyl-4H-1,2,4-triazol-3-yl)pyridine modification A is further characterized by an X-ray powder diffraction pattern essentially as shown in FIG. 1.

4-(5-{(1R)-1-[5-(3-chlorophenyl)isoxazol-3-yl]ethoxy}-4-methyl-4H-1,2,4-triazol-3-yl)pyridine modification A is a crystalline form exhibiting advantageous properties over the amorphous form, such as increased chemical and physical stability, lower hygroscopicity, higher purity, better yield and improved handling properties during manufacturing and post processing.

It is possible to crystallize 4-(5-{(1R)-1-[5-(3-chlorophenyl)isoxazol-3-yl]ethoxy}-4-methyl-4H-1,2,4-triazol-3-yl)pyridine modification A, i.e. the compound of the present invention in one single solvent or in a mixture of solvents.

Crystallization may be initiated and/or effected with or without seeding with crystals of the appropriate crystalline compound of the invention.

Crystallization of compounds of the present invention can be achieved starting from pure or a slurry of an amorphous form or from pure or a slurry of a salt of 4-(5-{(1R)-1-[5-(3-chlorophenyl)isoxazol-3-yl]ethoxy}-4-methyl-4H-1,2,4-triazol-3-yl)pyridine of any form, or mixtures of any forms.

In one embodiment of the present invention, 4-(5-{(1R)-1-[5-(3-chlorophenyl)isoxazol-3-yl]ethoxy}-4-methyl-4H-1,2,4-triazol-3-yl)pyridine modification A is obtained upon crystallization from acetonitrile.

In one embodiment of the present invention, 4-(5-{(1R)-1-[5-(3-chlorophenyl)isoxazol-3-yl]ethoxy}-4-methyl-4H-1,2,4-triazol-3-yl)pyridine modification A is obtained upon crystallization from dimethylsulphoxide and water.

One object of the present the invention is to provide a process for the preparation of 4-(5-{(1R)-1-[5-(3-chlorophenyl)isoxazol-3-yl]ethoxy}-4-methyl-4H-1,2,4-triazol-3-yl)pyridine modification A.

According to one embodiment 4-(5-{(1R)-1-[5-(3-chlorophenyl)isoxazol-3-yl]ethoxy}-4-methyl-4H-1,2,4-triazol-3-yl)pyridine, modification A, is prepared by recrystallizing amorphous 4-(5-{(1R)-1-[5-(3-chlorophenyl)isoxazol-3-yl]ethoxy}-4-methyl-4H-1,2,4-triazol-3-yl)pyridine in a solvent at a temperature of at least 65° C. Said solvent is preferably a boiling mixture of methanol and water.

Alternatively, other alcohols (e.g. ethanol, n-propanol, 2-propanol, n-butanol, tert-butanol) could be used as well as polar aprotic solvents (e.g. dimethylsulfoxide, N-Methyl Pyrrolidine, dimethyl formamide, acetonitrile) as single crystallization solvent or in any combination with or without water as co-solvent. Furthermore, esters (e.g. ethyl acetate, n-butyl acetate, isopropyl acetate), ethers (e.g. methyl tert-butyl ether, tetrahydrofurane, 2-methyl tetrahydrofurane 1,4-Dioxane) or ketones (e.g. acetone, methylethyl ketone, methyl iso-butyl ketone) may be considered as single crystallization solvent or any combination.

According to another embodiment, 4-(5-{(1R)-1-[5-(3-chlorophenyl)isoxazol-3-yl]ethoxy}-4-methyl-4H-1,2,4-triazol-3-yl)pyridine, modification A, is prepared by a process comprising the steps of:

a) mixing (R)-1-[5-(3-chloro-phenyl)-isoxazol-3-yl]-ethanol, 4-(5-methanesulfonyl-4-methyl-4H-[1,2,4]triazol-3-yl)pyridine and a base in a non-aqueous polar solvent;
b) heating the resulting mixture to at least 60° C. for at least 10 hours;
c) cooling the mixture to room temperature and adding water to the resulting mixture, thereby generating an aqueous phase and an organic phase; and
d) recovering crystalline 4-(5-{(1R)-1-[5-(3-chlorophenyl)isoxazol-3-yl]ethoxy}-4-methyl-4H-1,2,4-triazol-3-yl)pyridine from the organic phase.

In one embodiment, the non-aqueous polar solvent is chosen from the group of dimethylsulfoxide and dimethylformamide, N-methylpyrrolidone and acetonitrile. Most preferably, the solvent is dimethylsulfoxide.

In one embodiment, the base is selected from the group of caesium carbonate and potassium tert-butoxide.

It is also preferred that solvents selected from the group of methyl-tert-butyl ether, isopropyl acetate and ethyl acetate are added to the two-phase system obtained in the above mentioned step c), and that crystalline 4-(5-{(1R)-1-[5-(3-chlorophenyl)isoxazol-3-yl]ethoxy}-4-methyl-4H-1,2,4-triazol-3-yl)pyridine is obtained by slowly evaporating the organic phase.

Alternatively, crystalline 4-(5-{(1R)-1-[5-(3-chlorophenyl)isoxazol-3-yl]ethoxy}-4-methyl-4H-1,2,4-triazol-3-yl)pyridine according to claim 2 is added to the organic phase obtained in the above mentioned step c) in order to induce crystallization.

4-(5-{(1R)-1-[5-(3-chlorophenyl)isoxazol-3-yl]ethoxy}-4-methyl-4H-1,2,4-triazol-3-yl)pyridine modification A obtained according to the present invention is substantially free from other crystal and non-crystal forms of 4-(5-{(1R)-1-[5-(3-chlorophenyl)isoxazol-3-yl]ethoxy}-4-methyl-4H-1,2,4-triazol-3-yl)pyridine. The term “substantially free from other crystal and non-crystal forms of 4-(5-{(1R)-1-[5-(3-chlorophenyl)isoxazol-3-yl]ethoxy}-4-methyl-4H-1,2,4-triazol-3-yl)pyridine” shall be understood to mean that the desired crystal form of 4-(5-{(1R)-1-[5-(3-chlorophenyl)isoxazol-3-yl]ethoxy}-4-methyl-4H-1,2,4-triazol-3-yl)pyridine contains less than 15%, preferably less than 10%, more preferably less than 5% of any other forms of 4-(5-{(1R)-1-[5-(3-chlorophenyl)isoxazol-3-yl]ethoxy}-4-methyl-4H-1,2,4-triazol-3-yl)pyridine.

The crystal modification according to the present invention is useful for the prevention or treatment of gastroesophageal reflux disease, IBS, functional dyspepsia, cough, obesity, Alzheimer's disease, senile dementia, AIDS-induced dementia, Parkinson's disease, amyotrophic lateral sclerosis, Huntington's Chorea, migraine, epilepsy, schizophrenia, depression, anxiety, acute anxiety, obsessive compulsive disorder, ophtalmological disorders such as retinopathies, diabetic retinopathies, glaucoma, auditory neuropathic disorders such as tinnitus, chemotherapy-induced neuropathies, post-herpetic neuralgia and trigeminal neuralgia, tolerance, dependency, addiction and craving disorders, neurodevelopmental disorders including Fragile X, autism, mental retardation, schizophrenia and Down's Syndrome, pain related to migraine, inflammatory pain, chronic pain disorders, acute pain disorders, neuropathic pain disorders such as diabetic neuropathies, arthritis and rheumatitiod diseases, low back pain, post-operative pain, pain associated with various conditions including angina, renal or billiary colic, menstruation, migraine and gout, stroke, head trauma, anoxic and ischemic injuries, hypoglycemia, cardiovascular diseases and epilepsy.

It is further provided a pharmaceutical composition comprising the crystal modification according to the present invention, as active ingredient, in association with a pharmaceutically acceptable carrier, diluent or excipient and optionally other active pharmaceutical ingredients. The pharmaceutical compositions of this invention may be administered in standard manner for the disease condition that it is desired to treat, for example by oral, topical, parenteral, buccal, nasal, vaginal or rectal administration or by inhalation or insufflation. For these purposes the crystal modification according to the present invention may be formulated by means known in the art into the form of, for example, tablets, pellets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

In addition to the crystal modification according to the present invention, the pharmaceutical composition of this invention may also contain, or be co-administered (simultaneously or sequentially) with, one or more pharmacological agents of value in treating one or more disease conditions referred to herein.

Suitable daily doses of the compounds of formula I in the treatment of a mammal, including man are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

In the practice of the invention, the most suitable route of administration as well as the therapeutic dose will depend on the nature and severity of the disease to be treated. The dose, and dose frequency, may also vary according to the age, body weight and response of the individual patient.

The crystal modification according to the present invention may be further processed before formulation into a suitable pharmaceutical formulation. For example, the crystal modification may be milled or ground into smaller particles.

For the avoidance of doubt, “treatment” includes the therapeutic treatment, as well as the prophylaxis, of a condition.

The presence of additional substances in a sample, like pharmaceutical excipients, to be characterised by X-ray powder diffraction can mask some of the peaks in the above characterized crystal modification. This fact alone can of course not demonstrate that the crystal modification is not present in the sample. Under such circumstances due care must be used and the presence of substantially all main peaks in the X-ray powder diffraction pattern might suffice to characterize the crystal modification. It is thus preferred to analyse the crystal modifications of the present invention without the presence of additional substances.

According to a further aspect of the invention there is provided a method of treatment of a condition where 4-(5-{(1R)-1-[5-(3-chlorophenyl)isoxazol-3-yl]ethoxy}-4-methyl-4H-1,2,4-triazol-3-yl)pyridine modification A is required or desired, which method includes administering a therapeutically effective amount of the crystal modification according to the present invention to a patient in need of such treatment.

The crystal modification according to the present invention has the advantage that it is in a form that provides for increased chemical and physical stability, lower hygroscopicity, higher purity, better yield and improved handling properties during manufacturing and post processing, compared to the amorphous form. Contrary, to amorphous material which has a non-defined melting point the present crystal modification melts within a well defined range of 113-119° C. The skilled person will appreciate that factors such as purity and presence of solvents may affect the melting point.

The crystal form that crystallizes is related to the kinetics and equilibrium conditions of the respective crystal modification at the specific conditions. Thus, as may be appreciated by the skilled person, the crystal modification that is obtained depends upon both the kinetics and the thermodynamics of the crystallization process. Under certain thermodynamic conditions (solvent systems, temperature, pressure and concentration of compound of the invention), one crystal modification may be more stable than another (or indeed any other). However, crystal modifications that have a relatively low thermodynamic stability may be kinetically favoured. Thus, in addition, kinetic factors, such as time, impurity profile, agitation, the presence or absence of seeds, etc may also influence which crystal modification that crystallizes.

The terms “pure” and “pure crystallized fractions” as disclosed herein, relates to 4-(5-{(1R)-1-[5-(3-chlorophenyl)isoxazol-3-yl]ethoxy}-4-methyl-4H-1,2,4-triazol-3-yl)pyridine, modification A having a purity of at least 90% (wt).

The invention is illustrated, but in no way limited, by the following examples.

### EXAMPLES

### General

X-ray powder diffraction analysis (XRPD) was performed on samples prepared according to standard methods, for example those described in Giacovazzo, C. et al (1995), Fundamentals of Crystallography, Oxford University Press; Jenkins, R. and Snyder, R. L. (1996), Introduction to X-Ray Powder Diffractometry, John Wiley & Sons, New York; Bunn, C. W. (1948), Chemical Crystallography, Clarendon Press, London; or Klug, H. P. & Alexander, L. E. (1974), X-ray Diffraction Procedures, John Wiley and Sons, New York. X-ray analyses were performed using a PANalytical X'Pert Pro, Bragg-Brentano, θ-θ, Cu K_{α}, rotating sample.

XRPD distance values may vary in the range ±2 on the last decimal place.

It will be appreciated by the skilled person that XRPD intensities may vary when measured for essentially the same crystalline form for a variety of reasons including, for example, preferred orientation.

### Example 1

### Preparation of 4-(5-{(1R)-1-[5-(3-chlorophenyl)isoxazol-3-yl]ethoxy}-4-methyl-4H-1,2,4-triazol-3-yl)pyridine modification A

18.6 g 4-(5-{(1R)-1-[5-(3-chlorophenyl)isoxazol-3-yl]ethoxy}-4-methyl-4H-1,2,4-triazol-3-yl)pyridine was dissolved in 100 ml boiling methanol. To this solution, while boiling, was slowly added 100 ml water. Towards the end of the addition a cloudy mixture had formed. The mixture was allowed to come to room temperature and was left under continuous stirring for 3 h before the crystals were filtered off and washed with water. Finally, the crystals were dried under vacuum over sicapent. 17.4 g product corresponding to an isolated yield of 93.7% was achieved.

### Example 2

### Preparation of 4-(5-{(1R)-1-[5-(3-chlorophenyl)isoxazol-3-yl]ethoxy}-4-methyl-4H-1,2,4-triazol-3-yl)pyridine modification A

(R)-1-[5-(3-chloro-phenyl)-isoxazol-3-yl]-ethanol and 4-(5-methanesulfonyl-4-methyl-4H-[1,2,4]triazol-3-yl)pyridine were obtained in accordance with the disclosure of WO2007/043939. 10 g (44.7 mmol) (R)-1-[5-(3-chloro-phenyl)-isoxazol-3-yl]-ethanol, 12.8 g (53.7 mmol) 4-(5-methanesulfonyl-4-methyl-4H-[1,2,4]triazol-3-yl)pyridine, and 14.6 g (44.7 mmol) caesium carbonate were dissolved/suspended in 50 ml anhydrous dimethylsulfoxide (DMSO). The mixture was heated to and kept at 60° C. during 20 h. The mixture was then heated to 70° C. and additional 2.9 g (8.9 mmol) caesium carbonate was added. After 5.5 h, the conversion was 97%. The mixture was cooled to room temperature while 210 ml water was added to the mixture during 14 h, which generated a phase separation into a liquid and an oil phase. The mixture was then mixed with 100 ml methyl tert-butyl ether, 50 ml isopropyl acetate and 30 ml ethyl acetate which generated two clear liquid phases that were separated. The organic phase was evaporated slowly after which the product crystallized. It was then washed twice with water and isolated. 12.8 g product, corresponding to an isolated yield of 75% was achieved.

### Example 3

### Preparation of 4-(5-{(1R)-1-[5-(3-chlorophenyl)isoxazol-3-yl]ethoxy}-4-methyl-4H-1,2,4-triazol-3-yl)pyridine modification A

10 g (44.7 mmol) (R)-1-[5-(3-chloro-phenyl)-isoxazol-3-yl]-ethanol was mixed with 12.8 g (53.7 mmol) 4-(5-methanesulfonyl-4-methyl-4H-[1,2,4]triazol-3-yl)pyridine, 14.6 g (44.7 mmol) caesium carbonate and 50 ml anhydrous dimethylsulfoxide (DMSO). The mixture was heated to and kept at 70° C. during 19 h. Additional 2.9 g (8.9 mmol) caesium carbonate was added. After 3.5 h, the conversion was >98%. 7 ml water was added to the mixture while it was cooled to room temperature. Crystallization was initiated by addition of 0.1% (wt) 4-(5-{(1R)-1-[5-(3-chlorophenyl)isoxazol-3-yl]ethoxy}-4-methyl-4H-1,2,4-triazol-3-yl)pyridine modification A. 20 minutes later, additional water (43 ml) was charged during 4 h and the slurry was stirred overnight. The product was filtered off and washed once with DMSO/water (1/1, v/v) and twice with water before drying at 40° C. under vacuum. 15.5 g product, corresponding to an isolated yield of 88%, was achieved.

### Example 4

### X-Ray Powder Diffraction (XRPD) Pattern of 4-(5-{(1R)-1-[5-(3-chlorophenyl)isoxazol-3-yl]ethoxy}-4-methyl-4H-1,2,4-triazol-3-yl)pyridine modification A

The crystallized fractions obtained in examples 1-3 showed to be pure 4-(5-{(1R)-1-[5-(3-chlorophenyl)isoxazol-3-yl]ethoxy}-4-methyl-4H-1,2,4-triazol-3-yl)pyridine modification A. Modification A may be identified by the X-ray power diffraction (XPRD) pattern in the table below as well as in FIG. 1.

| d-spacing value (Å) | Relative intensity |
|---|---|
| 12.63 | Weak |
| 11.41 | Weak |
| 6.35 | Strong |
| 6.21 | Medium |
| 5.72 | Strong |
| 5.08 | Strong |
| 4.0 | Very strong |

d-value: the spacing between successive parallel hkl planes in a crystal lattice

The peaks, identified with d-values calculated from the Bragg formula and intensities, have been extracted from the diffractogram of 4-(5-{(1R)-1-[5-(3-chlorophenyl)isoxazol-3-yl]ethoxy}-4-methyl-4H-1,2,4-triazol-3-yl)pyridine modification A, shown in FIG. 1. The relative intensities are less reliable and instead of numerical values the following definitions are used:

| % Relative Intensity* | Definition |
|---|---|
| 25-100 | Very strong |
| 10-25 | Strong |
| 3-10 | Medium |
| 1-3 | Weak |

*The relative intensities are derived from diffractograms measured with variable slits.

1. 4-(5-{(1R)-1-[5-(3-chlorophenyl)isoxazol-3-yl]ethoxy}-4-methyl-4H-1,2,4-triazol-3-yl)pyridine in crystalline form.

2. 4-(5-{(1R)-1-[5-(3-chlorophenyl)isoxazol-3-yl]ethoxy}-4-methyl-4H-1,2,4-triazol-3-yl)pyridine in crystalline form according to claim 1, characterized in providing an X-ray powder diffraction pattern exhibiting substantially the following main peak with d-values:
d-spacing value (Å)
4.0

3. 4-(5-{(1R)-1-[5-(3-chlorophenyl)isoxazol-3-yl]ethoxy}-4-methyl-4H-1,2,4-triazol-3-yl)pyridine in crystalline form according to claim 1, characterized in providing an X-ray powder diffraction pattern exhibiting substantially the following main peak with d-values:
d-spacing value (Å)
4.0
5.08
5.72
6.35

4. 4-(5-{(1R)-1-[5-(3-chlorophenyl)isoxazol-3-yl]ethoxy}-4-methyl-4H-1,2,4-triazol-3-yl)pyridine in crystalline form according to claim 1, characterized in providing an X-ray powder diffraction pattern exhibiting substantially the following main peak with d-values:
d-spacing value (Å)
4.0
5.08
5.72
6.21
6.35
11.41
12.63

5. 4-(5-{(1R)-1-[5-(3-chlorophenyl)isoxazol-3-yl]ethoxy}-4-methyl-4H-1,2,4-triazol-3-yl)pyridine in crystalline form as defined in claim 2, characterized in providing an X-ray powder diffraction pattern essentially as shown in FIG. 1.

6. A compound as defined in claim 1 for use in therapy.

7. A pharmaceutical formulation comprising the compound according to claim 1 in admixture with at least one pharmaceutically acceptable excipient.

8. (canceled)

9. A method of treatment or prevention of a mGluR5 receptor-mediated disorder, wherein:
the disorder is selected from gastroesophageal reflux disease, IBS, functional dyspepsia, cough, obesity, Alzheimer's disease, senile dementia, AIDS-induced dementia, Parkinson's disease, amyotrophic lateral sclerosis, Huntington's Chorea, migraine, epilepsy, schizophrenia, depression, anxiety, acute anxiety, obsessive compulsive disorder, ophtalmological disorders, auditory neuropathic disorders, chemotherapy-induced neuropathies, post-herpetic neuralgia and trigeminal neuralgia, tolerance, dependency, addiction and craving disorders, neurodevelopmental disorders pain related to migraine, inflammatory pain, chronic pain disorders, acute pain disorders, neuropathic pain disorders, low back pain, post-operative pain, pain associated with angina, renal or billiary colic, pain associated with menstruation, pain associated with gout, stroke, head trauma, anoxic and ischemic injuries, hypoglycemia, cardiovascular diseases and epilepsy; and
the method comprises administration of a therapeutically effective amount of a compound according to claim 1, to a patient suffering from the disorder.
